(19) United States
(12) Patent Application Publication
(10) Pub. No.: US 2011/0166395 A1
(43) Pub. Date: Jul. 7, 2011

(54) METHOD FOR THE PRODUCTION OF NANOCRYSTALLINE NICKEL OXIDES

(76) Inventors: Hans-Jörg Wölk, Rosenheim (DE); Alfred Hagemeyer, Bad Aibling (DE); Frank Grossmann, Munchen (DE); Silvia Neumann, Grosskarolinenfeld (DE); Gerhard Mestl, Munchen (DE)

(21) Appl. No.: 12/995,272

(22) PCT Filed: May 29, 2009
(86) PCT No.: PCT/EP2009/003881

§ 371 Date: Feb. 22, 2011

(30) Foreign Application Priority Data
May 30, 2008 (DE) 10 2880 026 209.9

(51) Int. Cl. C01G 53/04; C07C 29/141; C07C 1/20

(52) U.S. Cl. 568/881; 423/594.19; 585/638

(57) ABSTRACT

The present invention relates to a method for the production of nanocrystalline nickel oxides as well as the nickel oxides produced by the method according to the invention and the use thereof as catalyst following reduction to nickel metal, in particular for hydrogenation reactions.

The present invention relates to a method for the production of nanocrystalline nickel oxides as well as the nickel oxides produced by the method according to the invention and the use thereof as catalysts and precursors and components for catalysts, in particular for hydrogenation reactions.

A comparable catalyst is known to a person skilled in the art under the name Raney nickel. This is a nickel-aluminium alloy which is converted to the activated Raney nickel by dissolving out much of the aluminium with caustic soda solution. Due to the resulting porous structure and therefore large BET surface area, Raney nickel has a high catalytic activity, in particular during hydrogenation reactions. Commercially available Raney nickel has an average nickel surface area of up to 100 m²/g. However, a disadvantage when using Raney nickel is that, because of the large surface area and reactivity, it can decompose spontaneously and explosively in air. The use of Raney nickel is therefore problematic in particular when used on an industrial scale.

Instead of Raney nickel as catalyst, it is also possible to use nickel oxide, which can be converted into an active nickel catalyst by reduction, as precursor. Unfortunately, nickel oxide, which is produced according to methods known in the state of the art has too small a BET surface area, with the result that the catalytic activity of the nickel which is obtained from the nickel oxide by reduction is frequently inadequate for chemical conversions.

The object of the present invention was therefore to provide a method in which nickel oxide with as large as possible a BET surface area and high catalytic activity (after reduction to nickel metal) can be obtained. The method is also to be easy to carry out and inexpensive.

The object is achieved by a method for the production of nanocrystalline nickel oxide material, comprising the steps of
a) the introduction of a nickel starting compound into a reaction chamber by means of a carrier fluid, wherein the nickel starting compound is a salt of an organic acid and wherein the nickel starting compound is introduced into the reaction chamber in the form of a solution, slurry, suspension or in solid aggregate state,
b) a thermal treatment of the nickel starting compound in a treatment zone by means of a pulsating flow at a temperature of from 200 to 550° C.,
c) the formation of nanocrystalline nickel oxide material,
d) the discharge of the nanocrystalline nickel oxide material obtained in steps b) and c) from the reactor.

It was surprisingly found that the method can be carried out at relatively low temperatures of from 200 to 550° C., particularly preferably from 230 to 500° C., particularly preferably from 250 to 480° C. Hitherto, preferred temperatures of more than 700° C., and indeed up to 1400° C., were known in the state of the art. Quite particularly surprisingly, it was also found that the crystallization process of the nickel oxide, in particular the size of the crystallites and the pore-size distribution of the corresponding nickel oxide can be controlled in targeted manner by the method according to the invention. This can further be advantageously influenced by the residence time in the flame or by the reactor temperature. The nanocrystalline nickel oxide particles that form are prevented from agglomerating by the pulsating thermal treatment. Typically, the nanocrystalline particles are immediately transferred through the stream of hot gas into a colder zone, where the nickel oxide crystallites are obtained, some with diameters of less than 20 nm.

In the case of the thus-obtainable nickel oxide crystallites, this leads to very high BET surface areas of >50 m²/g, particularly preferably >100 m²/g and particularly preferably >150 m²/g. In particular, nickel oxides with a BET surface area of up to 350 m²/g, preferably of from 200 to 300 m²/g were able to be obtained according to the method according to the invention. The BET surface area is determined according to DIN 66132 (using the Brunauer, Emmett and Teller method).

It proved to be advantageous if the nickel starting material is ground to a particle diameter of <10 μm, preferably <5 μm, particularly preferably <2 μm and in particular <1 μm. The particle size is preferably determined by the Debye-Scherrer method in conjunction with X-ray diffraction and a Rietveld refinement.

The method developed by Peter Debye and Paul Scherrer and also, independently of them, by Albert Hull, operates, not with monocrystals, but with powdery samples. The powder consists of a series of randomly arranged crystallites, with the result that the lattice planes are also arranged randomly in space and thus some crystallites always satisfy the Bragg reflection condition. In addition, the sample rotates about an axis perpendicular to the incident beam. Around the sample, cone-shaped shells form from X-rays which originate in the structural interference. A photographic film, on which the cone-shaped shells appear as reflexes, lies around the sample. The grazing angle θ can be calculated from the distances between the reflexes recorded on the film from the incident beam:

x/2πR=4θ/360°

The distance x of the diffraction reflex on the film from the incident beam behaves with respect to the circumference of the camera x/2πR like the aperture angle of the corresponding diffraction cone with respect to 360°. Regarding the X-ray diffractometric Rietveld analysis, we also refer to R. Kriegel, Ch. Kaps, Thüringer Werkstofftag “X-ray diffraktometrische Rietveld-Analyse von nanokristallinen Precursoren and Keramiken”, Verlag Dr. Köster, Berlin 2004, pages 51-56, the disclosure of which is incorporated herein by reference.

A smaller particle size of the nickel starting material leads to a further increase in the specific surface area of the nickel oxide obtained according to the invention, wherein in contrast the residual carbon content decreases. This is due to the more rapid transport of heat into the inside of the particle during the conversion in the pulsating fluidized-bed reactor and thus the creation of the necessary conditions for a reaction conversion also in the particle itself and not just in the outer shell region.

The preferred particle size is preferably set by wet grinding, i.e. by grinding a suspension of the nickel starting compound in a dispersant. The grinding can for example take place in a ball mill, bead mill, beater mill, an annular gap mill or other mills known in the state of the art. A pretreatment of the suspension by means of a dispersant (for example Ultra-Turrax T50) prior to the grinding also proved advantageous.

In the method according to the invention, suspensions can be calcined within a very short period, typically within a few milliseconds, at comparatively lower temperatures than are usual with methods of the state of the art, without additional filtration and/or drying steps or without the addition of additional solvents. The nickel nanocrystallites that form have significantly increased BET surface areas and thus, after a reduction to nickel metal, represent a nickel catalyst with increased reactivity, improved rate of conversion and improved selectivity.

The nearly identical residence time of every nickel oxide particle in the homogeneous temperature field created by the method results in an extremely homogeneous end product with narrow monomodal particle distribution. A device for carrying out the method according to the invention in the production of such monomodal nanocrystalline metal oxide powders is known for example from DE 101 09 892 A1. Unlike the device described there and the method disclosed there, the present method does not, however, require an upstream evaporation step in which the starting material, i.e. the nickel starting compound, is heated to an evaporation temperature.

The nickel starting compound from which the nickel oxide materials according to the invention are produced are inserted direct via a carrier fluid, in particular a carrier gas, preferably an inert carrier gas, such as for example nitrogen, etc., into so-called reaction chambers, i.e. into the combustion chamber. Attached exhaust side to the reaction chamber is a resonance tube with a flow cross-section which is clearly reduced compared with the reaction chamber. The floor of the combustion chamber is equipped with several valves for the entry of the combustion air into the combustion chamber. The aerodynamic valves are fluidically and acoustically matched to the combustion chamber and the resonance tube geometry such that the pressure waves, created in the combustion chamber, of the homogeneous “flameless” temperature field slide pulsating predominantly in the resonance tube. A so-called Helmholtz resonator forms with pulsating flow with a pulsation frequency of between 3 and 150 Hz, preferably 10 to 110 Hz.

Material is typically fed into the reaction chamber either with an injector or with a suitable two-component nozzle, or in a Schenk dispenser.

Preferably, the nickel starting compound is introduced into the reaction chamber in atomized form, with the result that a fine distribution in the region of the treatment zones is guaranteed.

A salt of an organic acid is preferably used as nickel starting compound, wherein an acid with at least one carboxyl group is preferably used as organic acid. According to the invention a salt of carbonic acid, i.e. a carbonate, e.g. Ni(OH)₂CO₃, is also to be regarded as salt of an organic acid.

A salt which has fewer than 9, preferably fewer than 8, particularly preferably fewer than 7 carbon atoms is preferably used as organic acid. It is quite particularly preferable if glyoxylic acid or oxalic acid is used as organic acid. Most preferred is the organic acid oxalic acid. It is furthermore preferred if the nickel starting compound is nickel carbonate (nickel salt of carbonic acid) or basic nickel carbonate (as suspension, paste or solution).

In addition to the nickel starting compound further compounds, for example support materials or precursors thereof, binders and/or promoters, can also be atomized simultaneously with the nickel starting compound. For example, an aluminium compound can advantageously also be atomized in order to obtain a nickel-aluminium system which can be converted into a nickel-aluminium catalyst (e.g. comparable with Raney nickel) by reduction. It is also possible for example to atomize aluminium nitrate with a nickel starting compound, wherein through the calcining in the pulsation reactor a nickel oxide supported on aluminium oxide can be obtained.

Other elements or compounds can also be used in the method according to the invention, in particular promoters, preferably selected from Al, W, Pd, Pt, Rh, Ru, Ag, Nb, Cu, Cr, Co, Mo, Fe and/or Mn. The promoters are preferably atomized and converted in the form of their salts together with the nickel starting material in the pulsation reactor. Following the production of the nickel oxide, the promoters can, however, also be introduced into the nickel oxide material in conventional manner, for example through a metal exchange or impregnation. Nickel-containing mixtures or mixed compounds can be obtained very simply in the ways mentioned above.

After the thermal treatment, the nanocrystalline nickel oxides (or nickel-containing mixtures or mixed compounds) that have formed are immediately transferred into a colder zone of the reaction chamber, if possible by means of the carrier fluid, with the result that they can be separated in the colder zone and discharged. The yield of the method according to the invention is almost 100%, as all of the product that forms can be discharged from the reactor.

Typically, the method is carried out at a pressure in the range of from normal pressure to approximately 40 bar.

A subject of the invention is furthermore the nanocrystalline nickel oxide material (or nickel-containing mixture or mixed compound) that can be obtained by the method according to the invention. It was found that the thus-obtainable nanocrystalline nickel oxide material preferably has a crystallite size in the range of from 4 nm to 100 μm, more preferably from 5 nm to 50 μm, quite particularly preferably 6 to 100 nm, which, as already stated above, can preferably be set by the pulsation of the thermal treatment. The particle size can be determined by XRD or TEM.

Furthermore, nickel oxide particles which have a BET surface area of preferably >50 m²/g, particularly preferably >100 m²/g and particularly preferably >150 m²/g are obtained by the method according to the invention. In particular, nickel oxides with a BET surface area of up to 350 m²/g, preferably from 200 to 300 m²/g, were able to be obtained according to the method according to the invention. In the process, the residual carbon content falls to ≦50 wt.-%, preferably to ≦20 wt.-%. Particularly preferably, the residual carbon content is ≦7.5 wt.-%, still more preferably ≦3 wt.-% and in particular ≦1 wt.-%.

An advantage of the nickel oxide material according to the invention is that, after reduction to nickel metal, it can be used to replace Raney nickel and is at a much smaller risk of exploding. It is therefore extremely suitable for use on an industrial scale. After reduction to nickel metal, the nickel oxide material according to the invention is pre-eminently suitable as hydrogenation catalyst, for example for the conversion or reduction of multiple-bond components, such as for example alkynes, alkenes, nitrides, polyamines, aromatics and substances of the carbonyl group. In addition, after reduction to nickel metal, heteroatom-heteroatom bonds of organic nitro compounds, for example nitrosamines, can be reduced with the nickel oxide compound according to the invention. The alkylation of amines, the amination of alcohols, a methanation, polymerization reactions or Kumada coupling represent further fields of use.

The nickel oxide material can be extruded with a suitable support material, for example aluminium oxide, and a suitable binder, for example boehmite or pseudoboehmite, to a shaped body. Likewise, an aluminium precursor, e.g. aluminium nitrate or peptized boehmite, which is converted together with the nickel starting compound according to the method according to the invention in the pulsation reactor, can also be used. A thus-produced nickel mixed oxide or the oxidic mixture can then immediately be compressed into a desired shape, for example into a simple tablet form. The NiO/Al₂O₃ molar ratio is preferably matched to that of conventional nickel hydrogenation catalysts and is preferably 60:40 to 40:60, preferably 55:45 NiO/Al₂O₃.

The invention will now be described in more detail with reference to the following embodiment examples, which are not to be understood as limiting. The device used, as already mentioned above, corresponds largely to the device described in DE 101 09 892 A1, with the difference that the device used for carrying out the method according to the invention had no preliminary evaporator stage.

### EMBODIMENT EXAMPLES

### Example 1

### Production of the Suspension

Nickel oxalate dihydrates (NiC₂O₄×2 H₂O) from two manufacturers, Molekula and Alfa Aesar respectively, were used as raw materials for this test. A suspension was produced from these raw materials as follows:

45 and 35 kg respectively of distilled water were added to 5 kg of nickel oxalate dihydrate from Molekula and 4 kg from Alfa Aesar respectively. The suspensions were mechanically pretreated by means of a disperser (Ultra Turrax T50) at 8,000 rpm for 4 minutes. The aim was to reduce the size of the particles. Both suspensions were combined and homogenized.

The average particle size (d₅₀) of the starting raw material nickel oxalate from the two suppliers differed. The nickel oxalate from Molekula had an average particle size of 3.7 μm and that from Alfa Aesar 9.1 μm. The quantity ratio of the two nickel oxalates used accordingly produces an average particle size of 6.1 μm in the suspension which was obtained by combining the two suspensions. However, an average particle size of 4.7 μm was ascertained for this combined, mechanically treated, suspension. The mechanical treatment of the suspension therefore led to a 1.4 μm reduction in the average particle size.

The settling of the solid in the suspension produced was prevented by stirring throughout the test operation.

### Example 2

### Production of Nickel Oxide from the Suspension

The suspension produced in Example 1 was sprayed into the heat treatment plant via a two-component nozzle with a feed quantity of 14 kg/h. Different process conditions were set for the respective test points.

The specific surface area and the total carbon concentration were determined on the sample material for the individual test points.

TABLE 1

| | | Specific | |
|---|---|---|---|
| | | surface area | |
| | Temperature | (according to | C_{tot.} |
| Test point | [° C.] | BET) [m²/g] | [wt.-%] |
| | 300 | 69 | — |
| 2 | 325 | 74 | — |
| 3 | 350 | 66 | 10.8 |
| 4 | 375 | 85 | 9.8 |
| 5 | 400 | 102 | — |
| 6 | 425 | 120 | 7.3 |

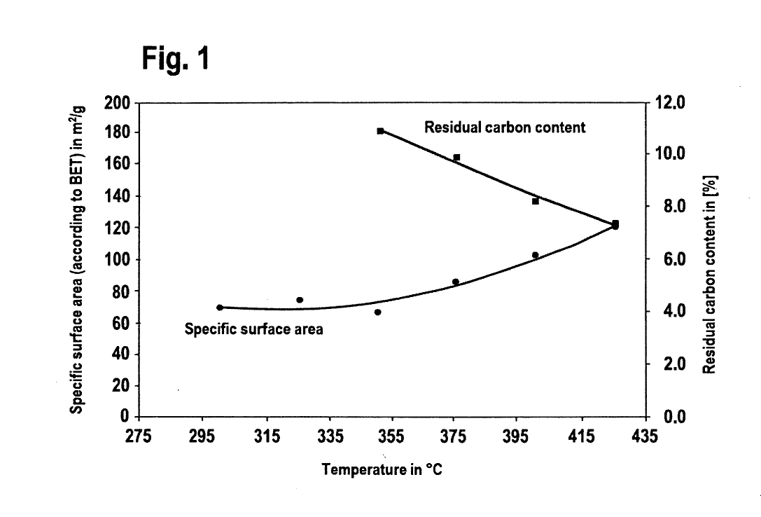

FIG. 1 shows the dependence of the specific surface area and the carbon concentration on the reaction temperature.

As the temperature rises, the carbon content continuously decreases, whereas the specific surface area increases. At 425° C. nickel oxide with a specific surface area of 120 m²/g and a residual carbon content of 7.3 wt.-% was obtained.

The necessary reactions for the thermal conversion of the nickel oxalate to nickel oxide require a necessary process temperature of the particle. As the heat transfer takes place from the hot gas to the particle and then by heat conduction from the surface of the particle into the inside of the particle, a heat gradient initially forms over the cross-section of the particle. The size of the gradient depends for example on the residence time and the particle diameter. A heat gradient over the particle diameter leads to different reaction conversions, and in special cases to a different intensity of the conversion of nickel oxalate.

Extending the residence time leads to a better reaction conversion in the inside of the particle, but cannot be achieved to the necessary extent on the pulsating fluid bed. A further rise in the process temperature would further reduce the residual carbon content in the product, as the decomposition of nickel oxalate proceeds, especially in the inside of the particle, but conceals the risk of sintering, particularly in the surface area. This would be associated with a reduction in the specific surface area.

### Example 3

### Reduction of the Particle Size of the Nickel Starting Compound

Grinding of the suspension produced in Example 1 in an annular gap mill (Fryma Koruma, Type MS 12) allowed the following reduction in average particle size:

TABLE 2

| Grinding process | Average particle size |
|---|---|
| 1st pass | 3.01 μm |
| 2nd pass | 2.96 μm |
| 3rd pass | 2.92 μm |
| 4rd pass | 2.65 μm |

### Example 4

### Production of Nickel Oxide from a Nickel Starting Compound with Reduced Particle Size

The suspension ground in Example 3 suspension was once more injected into the pulsating fluid bed with a feed quantity of 14 kg/h. The product had the following specification:

TABLE 3

| | | Specific | |
|---|---|---|---|
| | | surface area | |
| | Temperature | (according to | C_{tot.} |
| Test point | [° C.] | BET) [m²/g] | [wt.-%] |
| 7 | 450 | 165 | 3.9 |

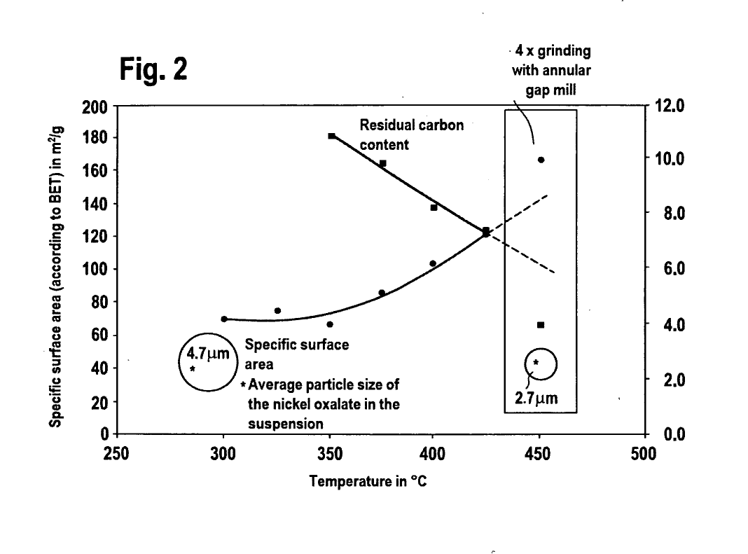

FIG. 2 shows the influence of the particle size on the specific surface area and the residual carbon content.

FIG. 2 shows that owing to the reduction in the average particle size of the nickel oxalate the specific surface area clearly increases, whereas the residual carbon content decreases.

This is due to the more rapid heat transport into the inside of the particle and hence the creation of the necessary conditions for a reaction conversion in the particle also, and not only in the outer shell region.

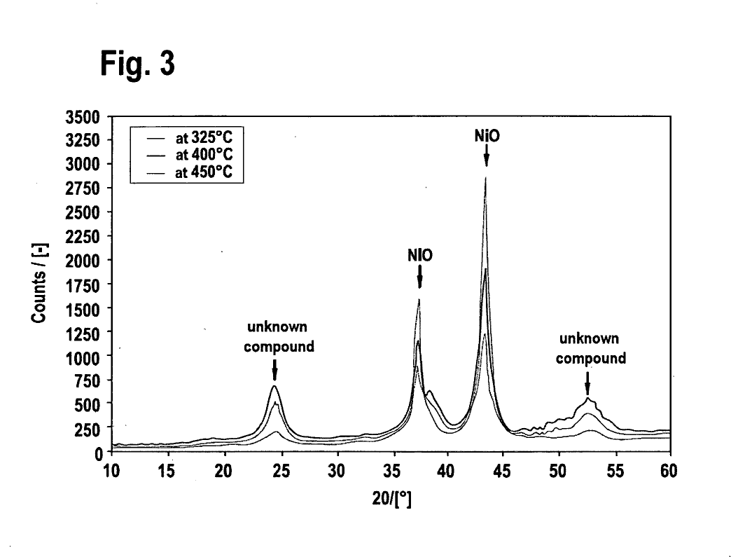

Selected samples of the nickel oxides produced were subjected to phase analysis by means of X-ray diffractometry (XRD). FIG. 3 shows the X-ray diffractograms. The following statements can be made on the basis of FIG. 3:

NiO was detected as crystalline phase in all the samples. The peak intensity increases as the treatment temperature rises, because of higher crystallinity.

Two other peaks could not be identified with the available database. However, it is assumed that these peaks are to be attributed to the nickel oxalate or one of its conversion products. This assumption is based on the fact that the intensity of these peaks decreases as the process temperature rises. This corresponds to the demonstrated course of the conversion of nickel oxalate to NiO.

### Example 5

### Production of Nickel Oxide from Nickel Oxalate with An Average Particle Size of <1 μM

12 kg of wet nickel oxalate with an average particle size of 5.4 μm was mechanically pretreated in order to achieve average particle sizes of <1 μm.

The wet nickel oxalate was mixed with distilled water to produce a 39 wt.-% nickel oxalate suspension and mechanically treated 2× in an annular gap mill (Fryma Koruma, Type MS 12):

TABLE 4
Grinding of nickel oxalate in the annular gap mill

| Grinding process | average particle size d₅₀ in μm |
|---|---|
| 1st pass | 0.84 |
| 2nd pass | 0.79 |

The average particle size of the nickel oxalate was thus reduced by approximately 4.6 μm.

Further distilled water was then added to this mechanically pretreated suspension, in order to obtain a 10 wt.-% nickel oxalate suspension and to provide conditions analogous to those in the previous test. The approach was similar as regards system parameters, i.e. no changes were made either to the material or to the equipment:
Material feed by means of a two-component nozzle,
Feed quantity 14 kg/h of suspension,
Stirring of the suspension in order to prevent settling.

A process starting temperature of 450° C. was fixed, which means a 10 K increase in the process temperature compared with the previous test.

The test results are summarized in Table 5:

TABLE 5

| | | Specific | |
|---|---|---|---|
| | Process | surface area | |
| | temperature | (according to | Total carbon |
| Test point | in ° C. | BET) in m²/g | C_{tot.} in wt.-% |
| 8 | 460 | 120 | 2.1 |
| 9 | 475 | 88 | 1.3 |
| 10 | 450 | 156 | 1.9 |

In test point 9 the process temperature was increased by another 15 K in order to further reduce the total carbon content. This was very clearly confirmed, but there is no reduction in the specific surface area here.

There are opposite effects on the total carbon and the specific surface area: the total carbon content steadily decreases as the process temperature rises, whereas the specific surface area of the nickel oxide passes through a maximum. The optimum process temperature lies within the range of from 450 to 460° C.

In order to investigate reproducibility with respect to the previous tests, a process temperature of 450° C. was again set in test point 10. In this test point the sampling was not carried out at the filter, but shortly after the exit from the reactor, in order to rule out confusion with test points 8-9.

The total carbon content, at 1.9 wt.-%, is 2% lower compared with the earlier test point (3.9 wt.-%). This is due to the smaller average particle size of the nickel oxalate in the suspension, which leads to a higher heat transfer into the inside of the particle and hence to an increased reaction conversion to NiO.

The oxidative conversion of nickel oxalate to nickel oxide takes place via intermediate stages. Nickel carbonate can be a possible transition compound. Advantages of nickel carbonate over nickel oxalate as raw material also include, in addition to economic aspects, more atom-efficient conversions (nickel content) to nickel oxide:

TABLE 6
Nickel content in nickel oxalate and nickel carbonate

| Raw material | Nickel content in % |
|---|---|
| Nickel oxalate dihydrate | 32 |
| Basic nickel carbonate | 58 |

### Example 6

### Production of Nickel Oxide from Nickel Carbonate

Basic nickel carbonate NiCO₃.2 Ni(OH)₂ from Aldrich was processed to produce a suspension. The average particle size of the solid is 5.4 μm. In order to guarantee identical molar ratios compared with the nickel oxalate suspension, the solids content of the nickel carbonate suspension was set at 16%. The nickel carbonate suspension was injected into the fluid bed at a process temperature of 460° C. In order to avoid confusion with the previous test points, sampling was again carried out after the exit from the reactor. The analysis results are summarized in Table 7:

TABLE 7
Test results of nickel oxide from basic nickel carbonate

| | | Specific | |
|---|---|---|---|
| | Process | surface area | |
| | temperature | (according to | Total carbon |
| Test point | in ° C. | BET) in m²/g | C_{tot.} in wt.-% |
| 11 | 460 | 62 | 0.2 |

The total carbon value of 0.2 wt.-% corresponds to the particularly preferred specification of the carbon content, which should preferably be less than 1 wt.-%. However, when basic nickel carbonate was used no mechanical pretreatment of the suspension was carried out.

### Example 7

### Reduction of the Process Temperature and Particle Size of the Starting Material

The basic nickel carbonate used (NiCO₃.2 Ni(OH)₂) has an average particle size of 5.4 μm. The complete thermal decomposition of such large raw material particles proves to be problematic in the pulsating fluidized bed because of the very short residence times. A complete conversion can be achieved only by increasing the process temperatures, wherein a more pronounced sintering thereby begins specifically in the region of the surface. This results in small specific surface areas.

Consequently, in this example also, the particle size of the raw material is to be reduced by grinding. Smaller particle sizes lead to a reduced temperature gradient into the inside of the particle and thus to a better reaction conversion at already lower process temperatures in the hot gas.

For this, distilled water was added to the basic nickel carbonate (NiCO₃.2 Ni(OH)₂ to form a 40 wt.-% nickel carbonate suspension and mechanically treated 3× in an annular gap mill (Fryma Koruma, Type MS 12):

TABLE 8
grinding of basic nickel carbonate in the annular gap mill

| | Average particle |
|---|---|
| | size d50 |
| Grinding process | [μm] |
| Start | 5.4 |
| 1^{st} pass | 1.7 |
| 2^{nd} pass | 1.1 |
| 3^{rd} pass | 0.8 |

The solids concentration of the suspension produced was then set at 16% by adding water.

The system configuration as well as the set process parameters likewise corresponded to the settings of the previous examples. The test material (suspension) was introduced into the reactor by fine-particle spraying by means of a two-component nozzle with a feed quantity of 14 kg/h of suspension. The raw material suspension was stirred throughout the test, in order to prevent settling.

A process starting temperature of 450° C. was fixed for the 1^{st} test point. The process temperature was then reduced in 25 K steps until the total carbon content rose to values of >1 wt.-%. The aim was to determine the optimum specific surface area. The test results are summarized in Table 9:

TABLE 9
Test results - NiO from basic nickel carbonate

| | | Specific | |
|---|---|---|---|
| | Process | surface area | |
| | temperature | (according to | Total carbon |
| Test point | [° C.] | BET) [m²/g] | C_{tot.} [wt.-%] |
| 1 | 450 | 77 | 0.3 |
| 2 | 425 | 84 | 0.4 |
| 3 | 400 | 94 | 0.5 |
| 4 | 375 | 121 | 0.9 |
| 5 | 350 | 134 | 1.4 |

As can be seen from Table 10, it was possible to obtain a fine-particled NiO with a specific surface area of 121 m²/g and a total carbon content of <1 wt.-%. The maximum of the specific surface area was 134 m²/g with a somewhat higher total carbon content of 1.4 wt.-%.

### Example 7-1

### Production of Nickel Oxide from Basic Nickel Carbonate Paste

Basic nickel carbonate paste Ni(OH)₂CO₃ from OMG Kokkola Chemicals OY was used as nickel starting compound. It was possible to obtain a nickel oxide with the following specifications with this paste:

Specific surface area according to BET: 244+/−5 m²/g
Total carbon: 1.0+/−0.05%
Average particle size: d50=13 μm
Colour: black
Crystallographic phase: crystalline (XRD)
X-ray diffractometer
Summary with Respect to the Production of NiO

Table 10 summarizes the results of the tests carried out starting from different raw materials to produce fine-particled NiO:

TABLE 10
Test results - NiO from different raw materials

| | | | Specific | |
|---|---|---|---|---|
| | average | Process | surface area | Total |
| | particle size | temperature | (according to | carbon |
| Raw material | d50 in μm | in ° C. | BET) in m²/g | C_{tot.} in wt.-% |
| Nickel | 2.7 | 450 | 165 | 3.9 |
| oxalate | | | | |
| suspension | | | | |
| Nickel | 0.8 | 450 | 156 | 1.9 |
| oxalate | | | | |
| suspension | | | | |
| Bas. nickel | 0.8 | 350 | 134 | 1.4 |
| carbonate | | | | |
| suspension | | | | |
| Bas. nickel | 13 | 400 | 244 + /− 5 | 1.0 +/− 0.05 |
| carbonate | | | | |
| paste | | | | |

All the tests were repeated, moreover with different promoters being used in different quantity ratios. The results are to be found in the “hydrogenation tests” section below.

The following statements can be made on the basis of the tests using different raw materials (nickel oxalate and basic nickel carbonate) to produce nickel oxide with high specific surface areas:

Basic nickel carbonate paste produced the highest specific surface areas with up to 350 m²/g (244 m²/g for approximately 1% residual carbon content). The production of fine-particled nickel oxide can also be carried out with nickel oxalate. At a process temperature of 450° C. and a nickel oxalate suspension with average particle sizes <1 μm an NiO with a specific surface area of 165 m²/g and a total carbon content of 3.9 wt.-% is obtained.

The tests were used to show the dependence of the specific surface area and the residual carbon on the process temperature. The reduction in the particle size of the nickel starting compound brought about a clear increase in the specific surface area of the nickel oxide and a reduction in the residual carbon content.

### Hydrogenation Tests

### Example 8

### Pore Distribution

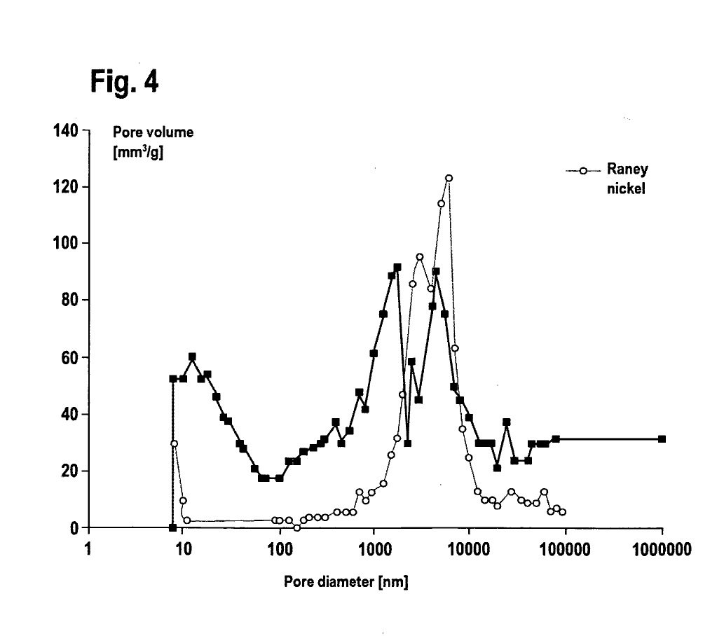

FIG. 4 shows the pore distribution of Raney nickel compared with the NiO produced in Example 7-1, which additionally contains 5 wt.-% W. The course of pore distribution shows that with Raney nickel there is increased concentration of the pores in ranges of from 1000 to 10000 nm. By contrast, the 5 wt.-% W/NiO catalyst has a relatively even distribution of pores over the spectrum.

### Example 9

### Hydrogenation of Octene

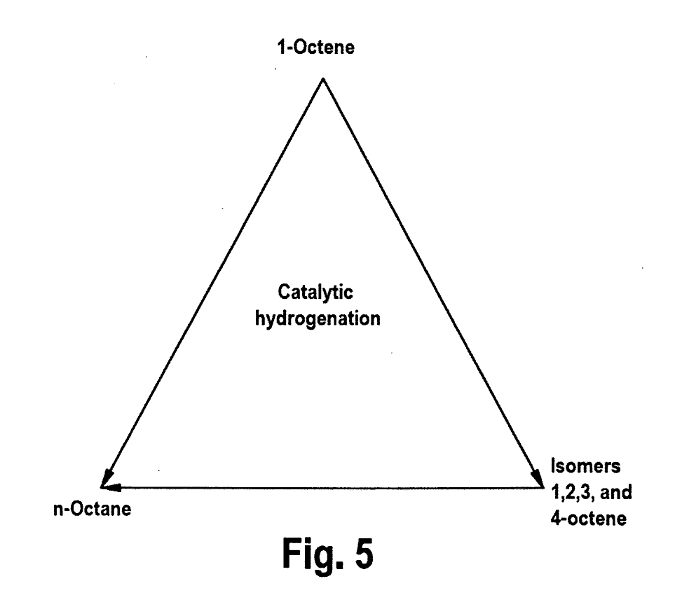

The hydrogenation of octene is used as example reaction for determining the catalytic activity between the undoped nickel oxides, doped nickel oxides and Raney nickel. According to this, a double bond shift (as shown in FIG. 5) takes place during the hydrogenation of octene. However, the double bond shift has no influence on the formation of octane, as the hydrogenation of the octene isomers takes place at the same reaction rate as the direct conversion of 1-octene to octane. Therefore the change in the concentrations of all the octene isomers is considered for the test evaluation.

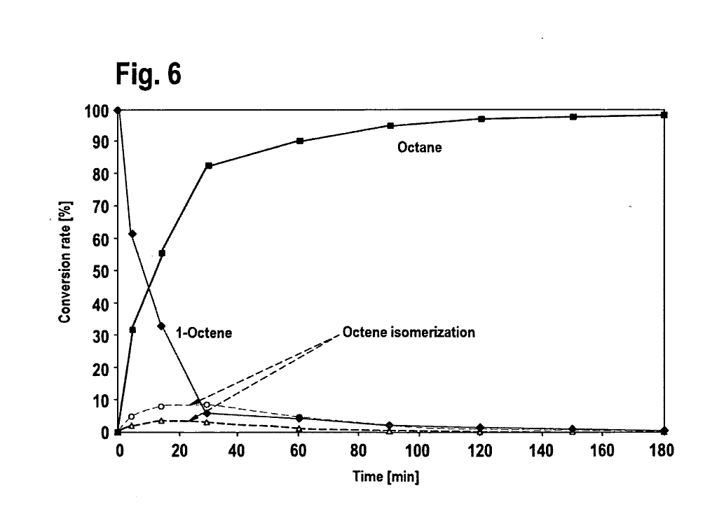

FIG. 6 shows the double bond shift as well as the hydrogenation reaction of octene and the octene isomers. The experimentally ascertained reaction mechanism corresponds precisely to the results found in the literature. There was no perceptible difference in the reaction mechanism of the catalysts used. Significant changes were recorded only for the reaction rate.

### Example 10

### Octene Hydrogenation with Different Pd-Doped Nickel Oxides Compared with Raney Nickel

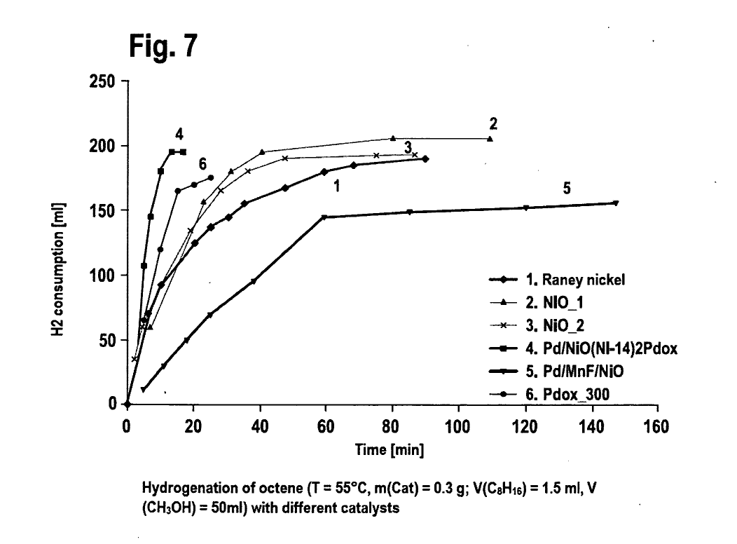

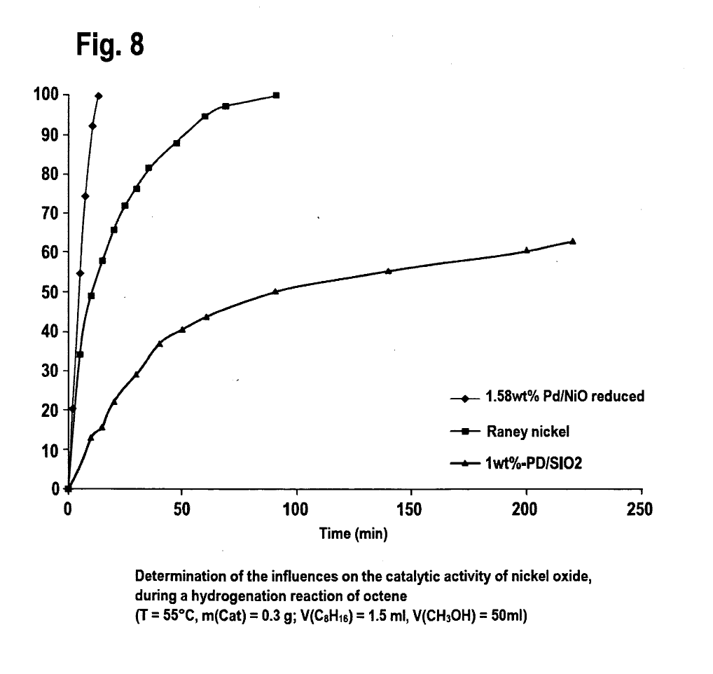

FIG. 7 shows the results of a hydrogenation of octene (T=55° C., m(Cat)=0.3 g; V(C₈H₁₆)=1.5 ml, V(CH₃OH)=50 ml) with different catalysts. FIG. 7 clearly shows that activated nickel oxide has a clearly better catalytic activity than Raney nickel. The addition of palladium served only to improve the hydrogen absorption during the reduction of the nickel oxide. The positive effect of palladium as promoter revealed itself during hydrogenation by a further marked increase in catalytic activity. As palladium is an equally known hydrogenation catalyst, the improvement in the catalytic activity of the doped nickel very probably resulted from the activity of the palladium, as can be seen in FIG. 8.

It was possible to demonstrate the influence of palladium on the catalytic activity during the hydrogenation reaction of octene by comparing a palladium catalyst on an SiO₂ support with the doped nickel oxide sample. The catalytic activity on the hydrogenation reaction of octene is clearly recognizable, so that the nickel oxide catalyst is the product of the activities of palladium and elementary nickel, which proved that the reduced catalyst has a very good catalytic activity.

### Example 11

### Octene Hydrogenation with Different Doped Nickel Oxides Compared with Raney Nickel

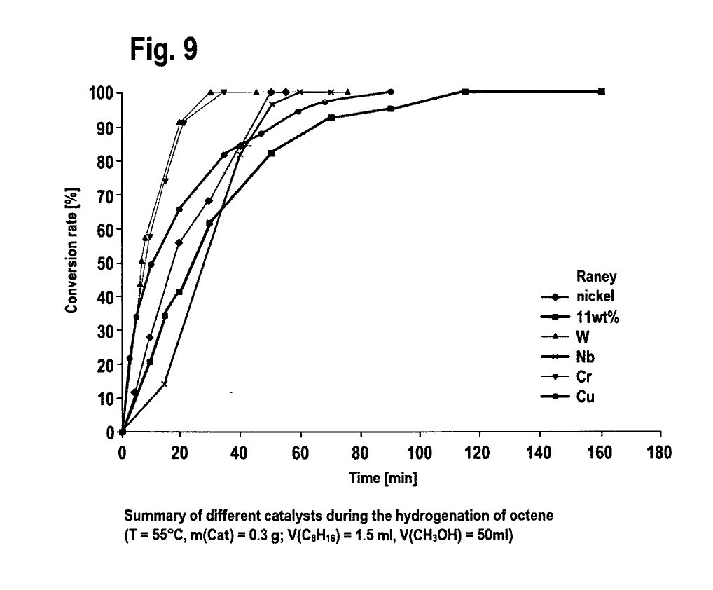

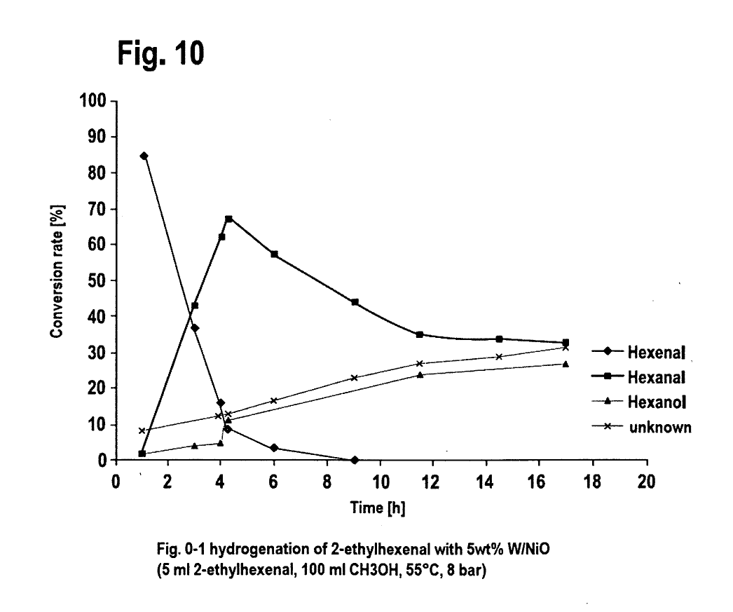

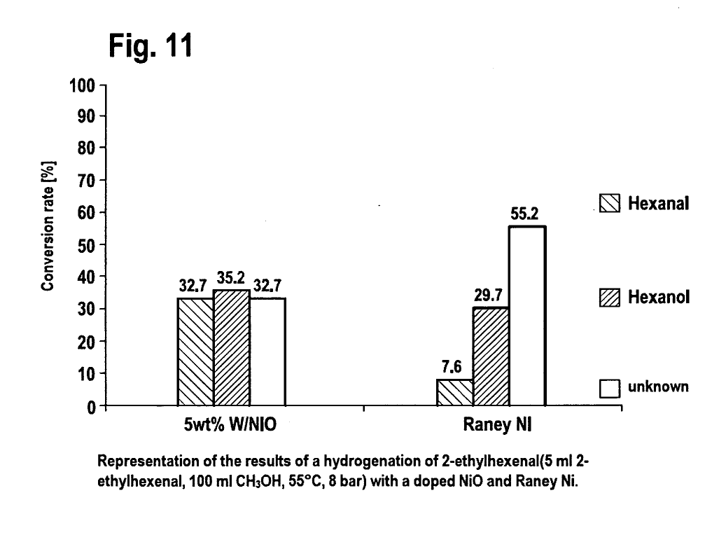

FIG. 9 shows the results of a comparison between an octene hydrogenation with different doped nickel oxides and a hydrogenation with Raney nickel. In the case of the hydrogenation of octene it can be said in summary that all the catalysts have a very good activity, but the catalysts doped with tungsten, niobium and chromium had a clearly improved catalytic activity compared with Raney nickel.

### Example 12

### Reaction Mechanism of 2-Ethylhexenal

Fresh knowledge was obtained when determining the reaction mechanism of 2-ethylhexenal.

The sequence of the hydrogenation of the double bond is of interest here. The hydrogenation reaction takes place according to the following equation:

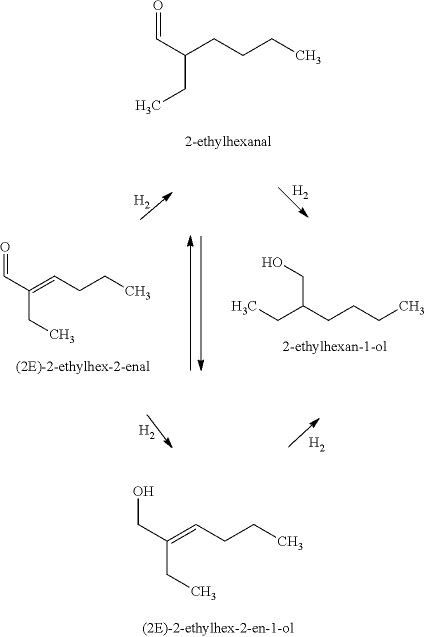

The following test parameters were weighed in and set:
5 ml 2-ethylhexenal,
100 ml CH₃OH,
0.3 g cat.
T 55° C.,
p=8 bar

The reaction mechanism of the hydrogenation was the same for both the catalysts used. However, there were significant differences with respect to the end-products, deactivation due to the solvent methanol presumably occurring in the case of the 5 wt.-% W/NiO catalyst. Raney nickel allows the hydrogenation of a hitherto still unknown substance to, a much greater extent than the 5 wt.-% W/NiO catalyst.

### Example 13

In the following a further doping of an NiO with aluminium hydroxide acetate hydrate (Example 7-1) was also carried out.

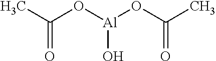

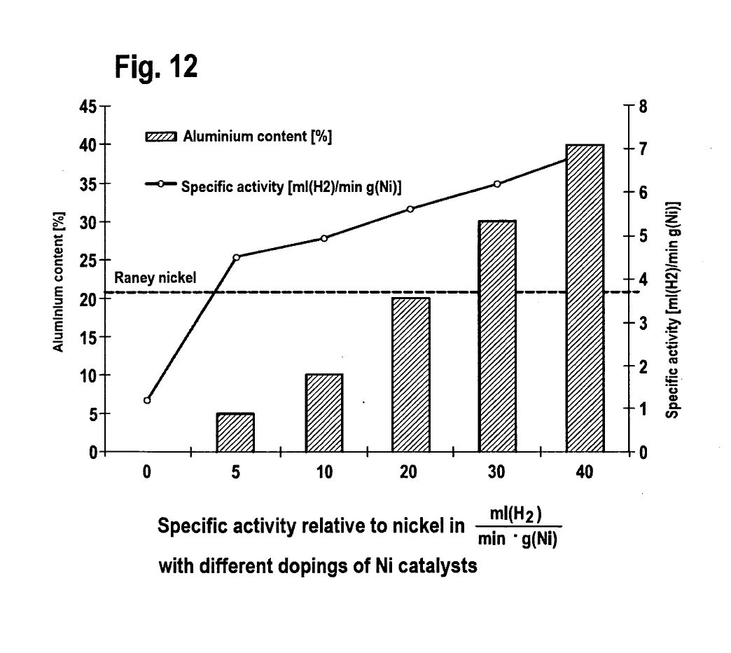

FIG. 12 clearly shows that, as the aluminium content increases, the specific activity of the catalyst increases, wherein at an aluminium content >20% the performance of Raney nickel is actually exceeded.

### Summary of the Results of the Hydrogenation Tests:

In summary, the produced nickel catalyst has a clearly better catalytic activity than the reference catalyst Raney nickel during the hydrogenation of a C═C double bond. Nickel oxide possesses advantages in respect of the reaction mechanism during reactions which can be catalyzed under slightly acid conditions, thus e.g. during the hydrogenation of 2-ethylhexenal. In terms of properties relevant to the surface area, nickel oxide possesses a surface area which is up to 100 times greater than that of Raney nickel. As regards pore distribution, a greatly broadened spectrum of 10-1,000,000 nm is encountered, wherein there is a concentration in pore frequency in the range of from 1,000 to 10,000 nm.

1. Method for the production of nanocrystalline nickel oxide material, comprising the steps
a) the introduction of a nickel starting compound into a reaction chamber by means of a carrier fluid, wherein the nickel starting compound is a salt of an organic acid and wherein the nickel starting compound is introduced into the reaction chamber in the form of a solution, slurry, suspension or in solid aggregate state,
b) a thermal treatment of the nickel starting compound in a treatment zone by means of a pulsating flow at a temperature of from 200 to 550° C.,
c) the formation of nanocrystalline nickel oxide material,
d) the discharge from the reactor of the nanocrystalline nickel oxide material obtained in steps b) and c).

2. Method according to claim 1, characterized in that the nickel starting compound has an average particle size of less than 10 μm.

3. Method according to claim 2, characterized in that the particle size is obtained by grinding a suspension of the nickel starting compound.

4. Method according to claim 1, characterized in that an acid with at least one carboxyl group is used as the organic acid.

5. Method according to claim 1, characterized in that the organic acid has fewer than 9 carbon atoms.

6. Method according to claim 1, characterized in that the organic acid is selected from glyoxalic acid, oxalic acid or derivatives thereof and carbonic acid (carbonate).

7. Method according to claim 1, characterized in that, in addition to the nickel starting compound, further compounds are also used in the method.

8. Method according to claim 7, characterized in that the further compounds are supports, binders and/or promoters.

9. Method according to claim 8, characterized in that Al, W, Pd, Pt, Rh, Ru, Ag, Nb, Cu, Cr, Co, Mo, Fe and/or Mn are used as promoters.

10. Nanocrystalline nickel oxide material according to claim 11, characterized by a BET surface area of more than 100 m²/g.

11. Nanocrystalline nickel oxide material obtained by a method according to claim 1.

12. Nanocrystalline nickel oxide material according to claim 11, characterized in that its crystallite size lies in the range of from 5 nm to 100 μm.

13. Nanocrystalline nickel oxide material according to claim 11, characterized in that it has a residual carbon content of less than 50 wt.-%.

14. A method for catalyzing a reaction, comprising the use of a nanocrystalline nickel oxide material according to claim 11 as catalyst or catalyst precursor for chemical conversions.

15. The method of claim 14, characterized in that the chemical conversion is a hydrogenation, a methanation, an alkylation of amines, an amination of alcohols, a polymerization reaction or a Kumada coupling.

16. The method of claim 14, wherein the nickel oxide material is reduced to metallic nickel.

17. Nickel catalyst obtained by reduction of the nanocrystalline nickel oxide material according to claim 11.
